(19) United States
(12) Patent Application Publication
(10) Pub. No.: US 2012/0220955 A1
(43) Pub. Date: Aug. 30, 2012

(54) Apparatus and Method for Preventing Aperture Re-Knitting

(75) Inventors: Luis Maseda, Natick, MA (US); Todd M. Chelak, Westborough, MA (US); AnnMarie L. Fisher, Winchendon, MA (US); Ian Kimball, Townsend, MA (US); William Siopes, Ayer, MA (US)

(73) Assignee: NP MEDICAL INC., Clinton, MA (US)

(21) Appl. No.: 13/035,427

(22) Filed: Feb. 25, 2011

(51) Int. Cl. A61M 5/00

(52) U.S. Cl. 604/256

(57) ABSTRACT

A cap for preventing re-knitting of an aperture within a proximal seal of a medical valve includes a body portion, a securing portion, and an interacting portion. The securing portion removably secures the cap to the inlet of the medical valve. The interacting portion interacts with the aperture to prevent the proximal seal from re-knitting.

### TECHNICAL FIELD

The present invention relates to medical valves. More specifically, the invention relates to preventing re-knitting of apertures within medical valves.

### BACKGROUND ART

In general terms, medical valving devices often act as a sealed port that may be repeatedly accessed to non-invasively inject fluid into (or withdraw fluid from) a patient's vasculature. During use, medical personnel may insert a luer tip syringe into the proximal port of a properly secured medical valve to inject fluid into (or withdraw fluid from) a patient. Once inserted, the syringe may freely inject or withdraw fluid to and from the patient.

It is well known that during sterilization (e.g., gamma irradiation) and storage of medical valves, the opposing surfaces of an aperture such as a slit may seal back together (e.g., they may “re-knit”). This re-knitting may hinder valve operation by making it difficult to open the aperture (e.g., upon connection of a medical implement). Some prior art valves use a lubricant to prevent the re-knitting. Other prior art valves utilize a shim or a tab that is inserted through the aperture and into the interior of the valve. However, each of the prior art methods to prevent re-knitting have significant drawbacks.

For example, by inserting a tab/shim through the aperture and into the inlet, there is a risk that a portion of the tab/shim may break off inside of the valve. As one would expect, this would significantly hinder the operation of the valve, and may render the valve inoperable. Additionally, because the shim extends through the aperture and physically keeps the aperture open, in many instances, the aperture within prior art valves may permanently deform and may no longer fully close. In such instances, there is a significant risk of contamination entering the interior of the valve.

### SUMMARY OF THE INVENTION

In accordance with one embodiment of the present invention, a system for preventing re-knitting of an aperture includes a medical valve and a cap. The medical valve may have an inlet and a proximal seal with a normally closed aperture through it. The cap may be secured to the inlet and have a body portion and a securing portion. The securing portion may secure the cap to the inlet. The body portion may have an interacting portion that interacts with the aperture to prevent the aperture from re-knitting. In some embodiments the securing portion may engage threads located on the valve inlet to secure the cap to the medical valve. The securing portion may also include a skirt that extends distally from the body portion and over the inlet of the medical valve.

The aperture may have a first aperture plane and a second aperture plane, and the interacting portion may deform the proximal seal to at least partially separate the first and second aperture planes when the cap is secured to the valve. The interacting portion may include a protrusion extending distally from the body portion, and the interacting portion may open the aperture when the cap is secured to the valve.

The system may also include a plurality of clips spaced about the securing portion. Each of the plurality of clips may have an engaging portion that engages threads located on the valve inlet and secures the cap to the valve. The plurality of clips may be configured to deform radially outward to allow the cap to slide over the inlet of the valve. Each of the plurality of clips may also have at least one living hinge that allows the clips to deform radially outward. The plurality of clips may be configured such that, when secured to the valve, the cap may be unthreaded from the medical valve to remove the cap from the medical valve.

The aperture may have a first and second aperture plane, and the interacting portion may urge the aperture planes away from one another as it interacts with the aperture. Additionally, the medical valve may include a valve mechanism within an interior of the valve. The valve mechanism may have an open mode which permits fluid flow through the valve and a closed mode that prevents fluid flow through the valve. The valve mechanism may remain in the closed mode as the interacting portion interacts with the aperture.

In accordance with other embodiments of the present invention, a cap for a medical valve may prevent re-knitting of an aperture within a proximal seal of the medical valve. The cap may include a body portion, a skirt, and a protrusion. The skirt may extend distally from the body portion and may be configured to fit over an inlet of the medical valve. The protrusion may extend distally from the body portion and interact with the aperture to prevent the aperture from re-knitting (e.g., the protrusion may urge the aperture planes away from one another as it interacts with the aperture).

The aperture may have a first aperture plane and a second aperture plane, and the protrusion may deform the proximal seal to at least partially separate the first and second aperture planes when the cap is connected to the valve. In some embodiments, the protrusion may open the aperture when the cap is secured to the valve.

The skirt may engage threads located on the valve inlet to secure the cap to the medical valve. Additionally, the cap may have a plurality of clips spaced about the skirt. The clips may have an engaging portion that engage the threads located on the valve inlet and secure the cap to the valve. The clips may be configured to deform radially outward to allow the cap to slide over the inlet of the valve. To that end, each clip may have at least one living hinge that allows the clip to deform radially outward so that the cap can slide over the inlet of the valve. The clips may also be configured to engage the threads such that, when secured to the valve, the cap may be unthreaded from the medical valve to remove the cap from the medical valve. The medical valve may include a valve mechanism within an interior of the valve. The valve mechanism may have an open mode which permits fluid flow through the valve and a closed mode that prevents fluid flow through the valve. The valve mechanism may remain in the closed mode as the protrusion interacts with the aperture and/or when the cap is secured to the inlet. The cap may have antimicrobial properties and/or include an antimicrobial swab that swabs the top of the valve as the cap is removed.

In accordance with additional embodiments of the present invention, a method for preventing re-knitting of an aperture within a medical valve includes providing a medical valve having an inlet housing, and securing a cap to the inlet housing. The medical valve may also have an inlet seal with an aperture, and a valve mechanism within an interior of the valve. The valve mechanism may be configured to transition the valve from a closed mode that prevents fluid flow through the valve to an open mode that permits fluid flow through the valve. The cap may include a body portion and a securing portion (e.g., a skirt) extending distally from the body portion and over the inlet housing. The body portion may also have a protrusion that interacts with the aperture to prevent the aperture from re-knitting. The securing portion may engage threads located on the inlet housing to secure the cap to the medical valve.

The aperture may have a first aperture plane and a second aperture plane, and the protrusion may deform the aperture to at least partially separate the first and second aperture planes when the cap is connected to the valve. In some embodiments, the protrusion may open the aperture when the cap is secured to the valve. In other embodiments, the protrusion may urge the aperture planes away from one another as it interacts with the aperture.

The cap may also include a plurality of clips spaced about the securing portion. Each of the clips may have an engaging portion that engages threads located on the valve inlet and secures the cap to the valve. The clips may be configured to deform radially outward to allow the cap to slide over the inlet of the valve. To that end, each of the clips may have at least one living hinge that allows the clip to deform radially outward. The clips may also be configured such that, when secured to the valve, the cap may be unthreaded from the medical valve to remove the cap from the medical valve. The medical valve may include a valve mechanism within the interior of the valve. The valve mechanism may have an open mode which permits fluid flow through the valve and a closed mode that prevents fluid flow through the valve. The valve mechanism may remain in the closed mode as the protrusion interacts with the aperture.

In accordance with still further embodiments, a system for preventing re-knitting of an aperture may include a medical valve and a cap. The medical valve may have an inlet and proximal seal with a normally closed aperture. The normally closed aperture, in turn, may include a first slit plane and a second slit plane. The cap may be removably secured to the inlet and may have a body portion and a securing portion. The securing portion may secure the cap to the inlet. The body portion may have an interacting portion that applies a radially outward force on the first and second slit planes to prevent re-knitting of the aperture. The radially outward force may or may not open the aperture when the cap is secured to the valve.

### BRIEF DESCRIPTION OF THE DRAWINGS

The foregoing features of the invention will be more readily understood by reference to the following detailed description, taken with reference to the accompanying drawings, in which:

FIG. 1 schematically shows one use of a medical valve configured in accordance with one embodiment of the present invention.

FIG. 2 schematically shows a perspective view of a medical valve configured in accordance with illustrative embodiments of the present invention.

FIG. 3 schematically shows a perspective view of a medical valve of FIG. 2 with an inlet cap in accordance to illustrative embodiments of the present invention.

FIG. 4 schematically shows a perspective view of the medical valve of FIG. 3 with the inlet cap secured to the valve in accordance with illustrative embodiments of the present invention.

FIGS. 5A-5C schematically show details of the inlet cap in accordance with illustrative embodiments of the present invention.

FIG. 6A schematically shows the medical valve of FIG. 2 with the inlet cap secured to the valve and a portion of the inlet housing in cross-section, in accordance with embodiments of the present invention.

FIG. 6B schematically shows the medical valve of FIG. 2 with the inlet cap secured to the valve and a portion of the inlet housing in cross-section, in accordance with alternative embodiments of the present invention.

FIG. 6C schematically shows the medical valve of FIG. 2 with the inlet cap secured to the valve and a portion of the inlet housing in cross-section, in accordance with an additional embodiment of the present invention.

FIG. 7 schematically shows a cross-sectional view of the valve shown in FIG. 4, in accordance with various embodiments of the present invention.

FIGS. 8A-8D schematically show an additional embodiment of an inlet cap in accordance with various embodiments of the present invention

### DETAILED DESCRIPTION OF SPECIFIC EMBODIMENTS

In illustrative embodiments, a cap placed over the inlet of a medical valve interacts with an inlet seal to prevent re-knitting of an aperture within the inlet seal (e.g., during sterilization and storage). Details of illustrative embodiments are discussed in greater detail below.

As mentioned above, sterilization (e.g., gamma irradiation) and storage of medical valves, can cause the opposing surfaces of an aperture such as a slit to seal back together (e.g., they may “re-knit”). As used herein, the term “re-knit” or “re-knitting” refers to the full or partial re-sealing of the aperture (e.g., the slit planes) such that the operation of the medical valve is hindered. Various embodiments of the present invention ensure proper operation of the valve by significantly reducing (or preventing) the re-knitting that occurs during sterilization and storage. In other words, although some nominal amount of re-sealing/re-knitting may occur when using some embodiments of the present invention, any nominal re-sealing/re-knitting may be overcome during normal operation of the medical valve (e.g., the aperture is still able to open after insertion of a medical implement into the valve inlet).

It is important to note that some re-knitting may occur after removal of the cap(s) discussed below. Therefore, care must be taken to limit the amount of time between removal of the cap and use of the valve, and limit exposure to high temperatures (e.g., the valve should be stored in ambient conditions after removal of the cap). Accordingly, it is recommended that the medical valve be stored in ambient conditions and used within a few days (preferably within a few minutes) after removal of the cap.

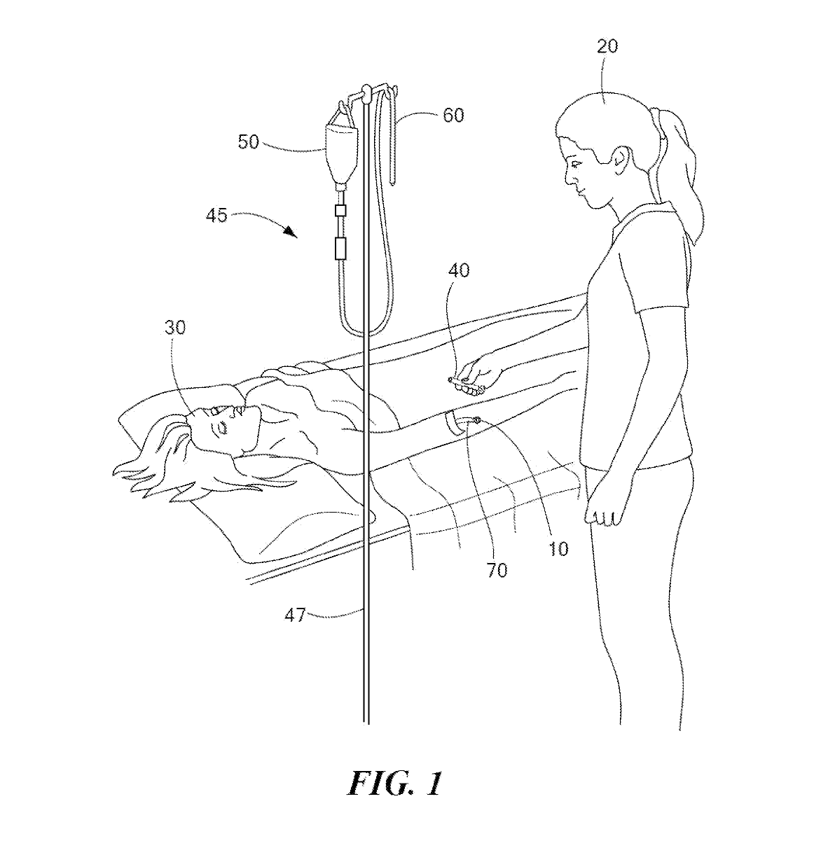

FIG. 1 schematically shows one illustrative use of a medical valve 10 configured in accordance with illustrative embodiments of the invention. In this example, a catheter 70 connects the valve 10 with a patient's vein (the patient is identified by reference number 30). Adhesive tape or similar material may be coupled with the catheter 70 and patient's arm to ensure that the valve remains in place.

After the valve 10 is in place, a nurse, doctor, technician, practitioner, or other user (schematically identified by reference number 20) may intravenously deliver medication to the patient 30, who is lying in a hospital bed. To that end, after the valve is properly primed and flushed (e.g., with a saline flush), the nurse 20 swabs the top surface of the valve 10 to remove contaminants. Next, the nurse 20 uses a medical instrument 40 (e.g., a syringe having a distally located blunt, luer tip complying with ANSI/ISO standards) to inject medication into the patient 30 through the valve 10. For example, the medical practitioner 20 may use the valve 10 to inject drugs such as heparin, antibiotic, pain medication, other intravenous medication, or other fluid deemed medically appropriate. Alternatively, the nurse 20 (or other user) may withdraw blood from the patient 30 through the valve 10.

The medical valve 10 may receive medication or other fluids from other means, such as through a gravity feed system 45. In general, traditional gravity feeding systems 45 often have a bag 50 (or bottle) hanging from a pole and containing a fluid (e.g., anesthesia medication) to be introduced into the patient 30. The medical practitioner 20 then connects the bag/bottle 50 to the medical valve 10 using tubing 60 having an attached blunt tip. In illustrative embodiments, the blunt tip of the tubing has a luer taper that complies with the ANSI/ISO standard. After the tubing 60 is connected to the medical valve 10, gravity (or a pump) causes the fluid to begin flowing into the patient 30. In some embodiments, the feeding system 45 may include additional shut-off valves on the tubing 60 (e.g., stop-cock valves or clamps) to stop fluid flow without having to disconnect the tubing 60 from the valve 10. Accordingly, the valve 10 can be used in long-term “indwell” procedures.

After administering or withdrawing fluid from the patient 30, the nurse 20 should appropriately swab and flush the valve 10 and catheter 70 to remove contaminants and ensure proper operation. As known by those skilled in the art, there is a generally accepted valve swabbing and flushing protocol that should mitigate the likelihood of infection. Among other things, as summarized above, this protocol requires proper flushing and swabbing before and after the valve is used to deliver fluid to, or withdraw fluid from the patient.

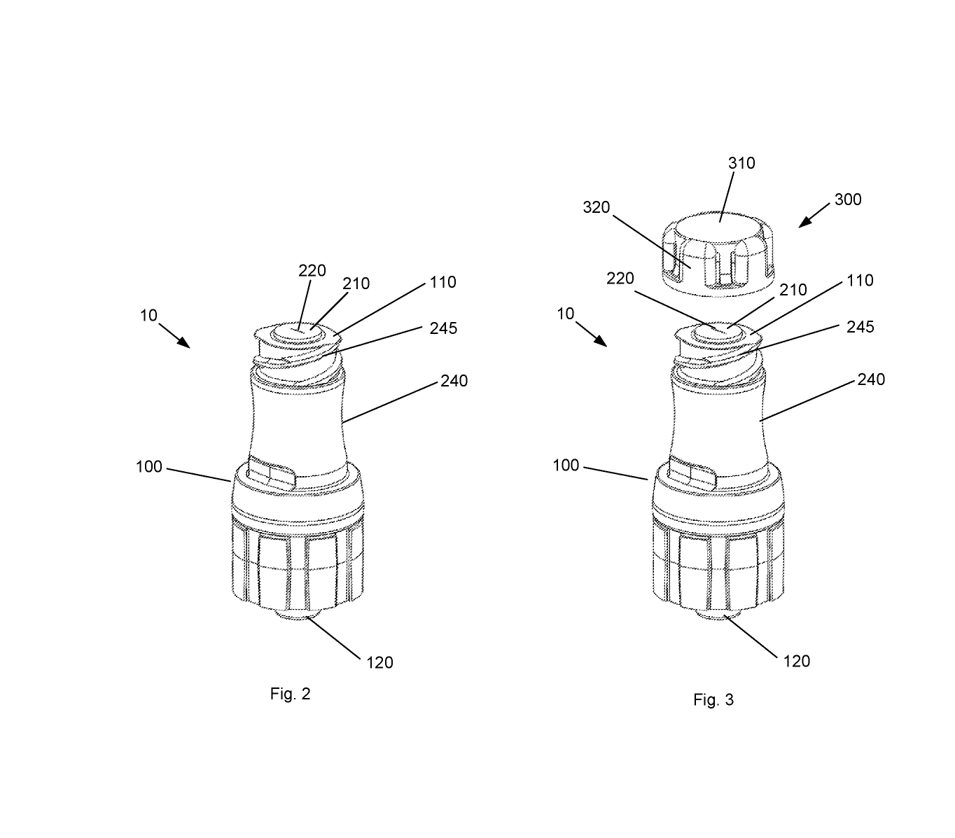

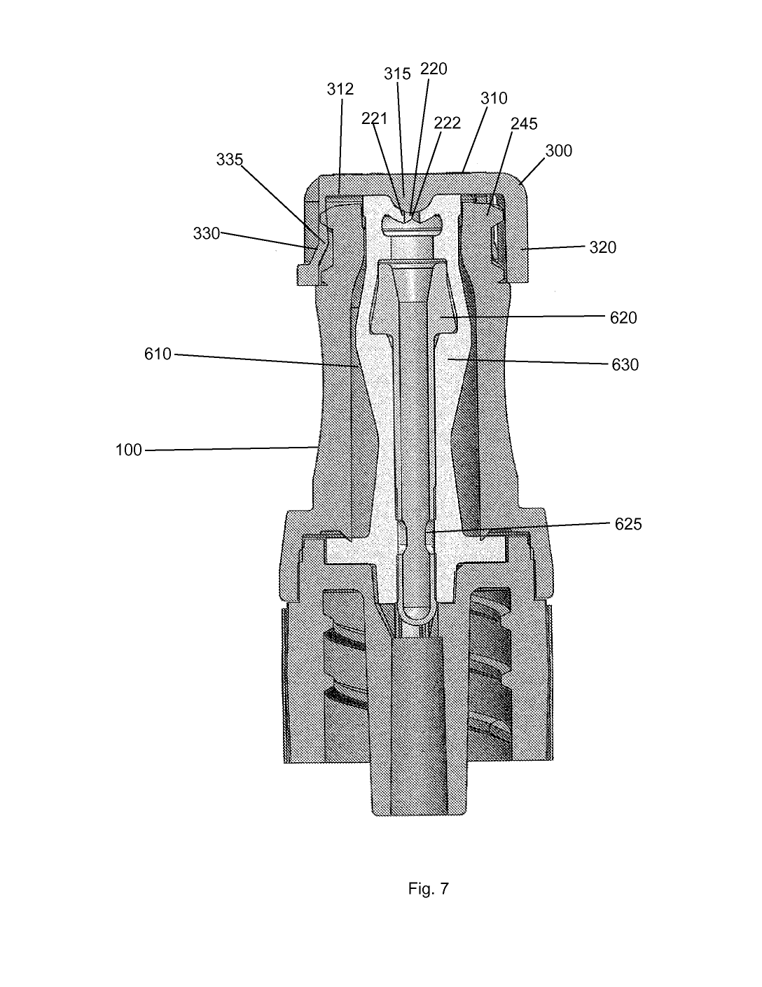

FIG. 2 schematically shows a perspective view of the medical valve 10 shown in FIG. 1. In illustrative embodiments, the medical valve 10 has a housing 100 forming an interior having a proximal port 110 for receiving the instrument 40 and a distal port 120 for injection or withdrawing fluids from the patient. The valve 10 has an open mode that permits fluid flow through the valve 10, and a closed mode that prevents fluid flow through the valve 10. To that end, the interior of the medical valve 10 may contain a valve mechanism 610 (FIG. 7, discussed below) that selectively controls fluid flow through valve 10.

The valve 10 may also have a resilient proximal gland 210 (e.g., an inlet seal). The resilient proximal gland 210 has a resealable aperture 220 that extends entirely through the proximal gland 210. The aperture 220 may, for example, be a pierced hole, or one or more slits (e.g., arranged into a cross). Alternatively, the proximal gland 210 may be molded with the aperture 220. As discussed in greater detail below, as the medical instrument 40 is inserted into the valve 10, the proximal gland 210 begins to deform and the aperture 220 opens, allowing the medical instrument 40 to enter the interior of the medical valve through the proximal port 110. In some embodiments, the medical instrument 40 does not need to penetrate the proximal gland 210. Rather, the medical instrument 40 may deform the proximal gland 210 enough to open the aperture 220, but not actually pass through the aperture 220.

It is also important to note that opening of the aperture 220 is not required for the medical instrument 40 to enter the valve 10. Opening of the aperture 220 is merely required to allow fluid transfer through the valve. For example, if the aperture 220 has re-knitted during sterilization/storage as described above, the medical instrument 40 may be connected to the valve 10 (e.g., the medical instrument 40 may enter the valve 10). However, because the aperture 220 has re-knitted and will not open, the practitioner 20 will be unable to transfer fluids through the valve 10 (e.g., they will be unable to administer medication to the patient 30).

As mentioned above, a medical practitioner may open the medical valve 10 by inserting a medical instrument 40 into the valve 10. In particular, when the medical instrument 40 makes contact with the inlet seal 210 and the medical practitioner 20 begins to move the instrument 40 distally, the inlet seal 210 will begin to deform. As the medical instrument 40 is inserted further, the inlet seal 210 will deform into the internal area of the medical valve 10 (e.g., it will deform into the area within the inlet housing 240). As the inlet seal 210 deforms, the aperture 220 opens creating fluid communication between the medical instrument 40 and the internal area of the housing. If the medical valve 10 has an internal valving mechanism 610 (FIG. 7), the tip of medical instrument 40 may pass through a portion of the inlet seal 210 and aperture 220 and actuate/open the internal valving mechanism.

As described above, the inlet seal 210 may be made from a resilient material (e.g. silicone) that allows the inlet seal 210 to automatically return back to the normal (e.g., at rest) shape in the absence of pressure/force. In other words, as the medical practitioner 20 removes the medical instrument 40, the inlet seal 210 will begin to return to the at rest position shown in FIG. 2. Additionally, as the instrument 40 is withdrawn, the aperture 220 will also close, fluidly disconnecting the medical instrument 40 with the internal area of the valve 10.

Some embodiments of the present invention may be swabbable. To that end, as best shown in FIG. 2, the inlet seal 210 may be substantially flush with or extend slightly proximal to the proximal port 110 when the valve 10 is in the closed mode. This creates a swabbable surface at the inlet of the valve 10 and allows the nurse 20 to perform the swabbing protocol discussed above. Other embodiments may not be swabbable. In such embodiments, the inlet seal 210 may be recessed from the proximal port 110.

After manufacturing and prior to use, medical valves like those described above are sterilized and stored. During the sterilization process and/or during storage the aperture 220 may re-seal. For example, if the aperture 220 is a slit, the slit planes (e.g., the opposing surfaces of inlet seal 210 through which the slit extends), which are in contact with each other during sterilization and storage, may adhere to one another or “re-knit” together. As discussed in greater detail below, if the slit planes re-knit together, it may be difficult to open the aperture and/or create the fluid communication needed for proper valve operation (e.g., because the aperture 220 may not open during valve actuation).

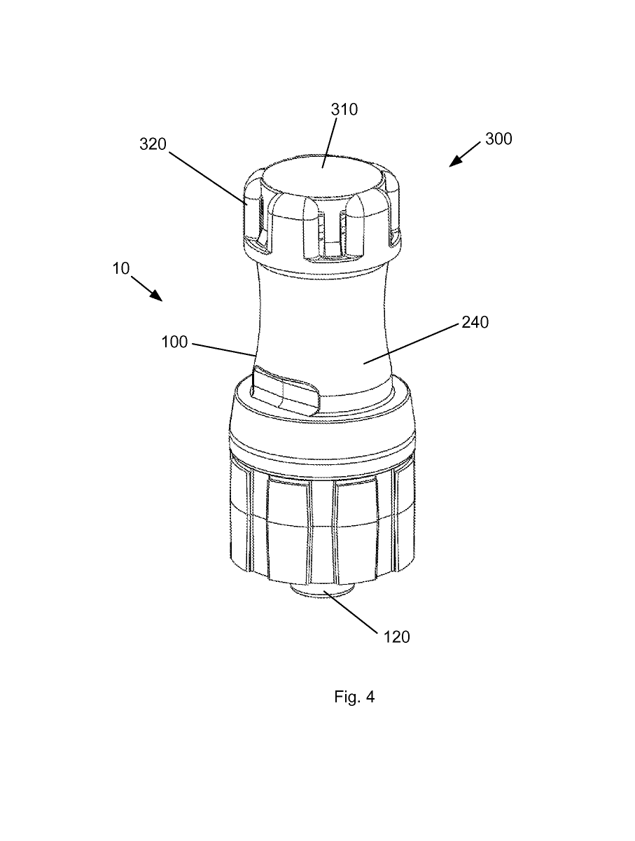

Various embodiments of the present invention mitigate and/or prevent the re-knitting process to ensure proper operation of the valve 10. To that end, as shown in FIGS. 3 and 4, some embodiments of the present may include a cap 300 that is placed over the valve inlet housing 240 prior to sterilization and during storage. The cap 300 may include a body portion 310 and a skirt 320 extending distally from the body portion 310, FIGS. 3, 4, and 5A-5C. As shown in FIG. 4, the body portion 310 sits above the inlet housing 240 and may rest on the inlet seal 210. The skirt 320 extends down over the inlet housing 240 and engages the threads 245.

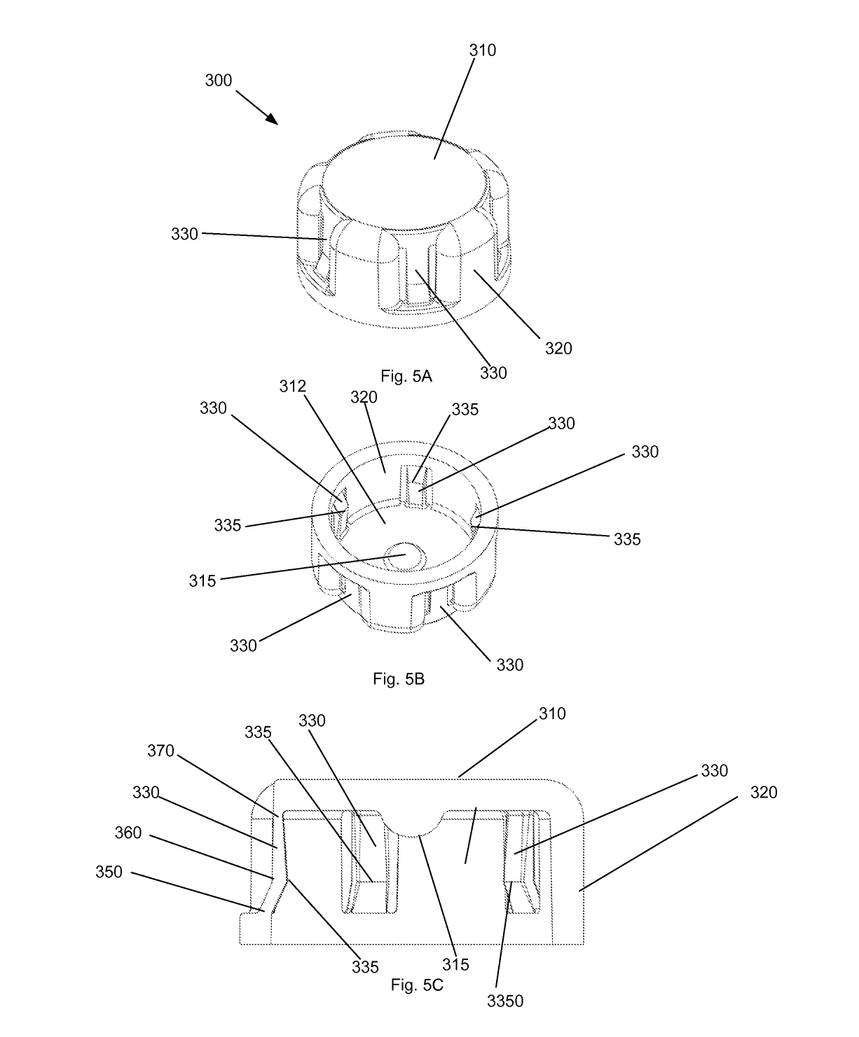

In order to facilitate engagement with the threads 245, the skirt 320 may have a number of clips 330 (FIG. 5B) spaced about the diameter of the skirt 320. As the cap 300 is slid over the inlet housing 240, the clips 330 may deform/flex radially outward to allow the cap 300 to fit over the threads 245. Once the cap 300 is in place, some of the clips 330 may at least partially return to their undeformed state and engage the threads 245. For example, the clips 330 may have an engagement portion 335 that sits under/engages one of the threads to secure the cap 300 in place and prevent the cap 300 from inadvertently falling off. Although some of the clips 330 may return to their undeformed state, others may remain deformed by the threads 245. For example, the engagement portion 335 of some of the clips 330 may be located at/on a thread 245 (e.g., instead of sitting under the thread 245) which, in turn, causes the clip 330 to remain deformed.

In order to allow sufficient deformation/flexing, the clips 330 may have one or more living hinges that flex as the cap 300 is placed on the valve 10. For example, each clip 330 may have three living hinges. The first living hinge 350 may be near the bottom of the skirt 320 and the clip 330, the second living hinge 360 may be located at the engaging portion 360, and the last living hinge 370 may be located at the top of the clip 330 where the skirt 320 meets the body portion 310. These “living hinges” may be thinned areas that allow the clips 330 to deform more easily at their respective locations.

Although the above described embodiments are described as having skirts 320, other embodiments may utilize different structures to secure the cap 300 to the valve 10. For example, some embodiments may have a plurality of legs (not shown) extending distally from the body portion 310. In such embodiments, the clips 330 may be located on the legs. In further embodiments, the cap 300 may have neither a skirt nor legs and only have the clips 330. For example, the clips 330 may be attached directly to the body portion 310 and extend distally from the body portion 310.

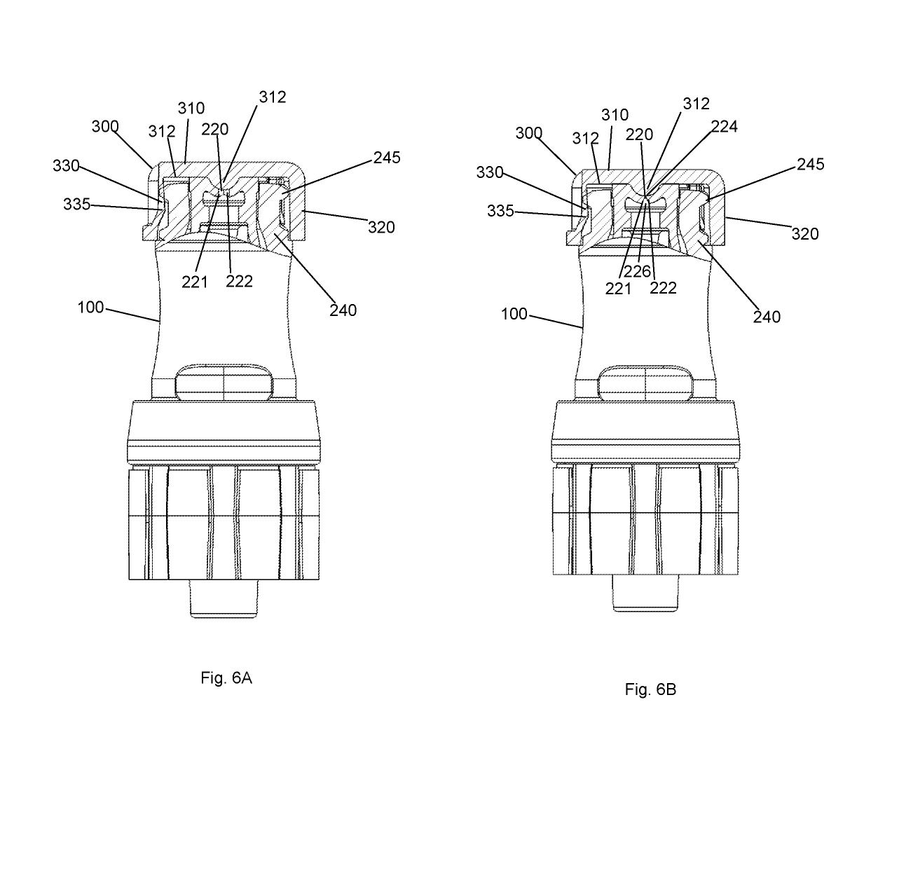

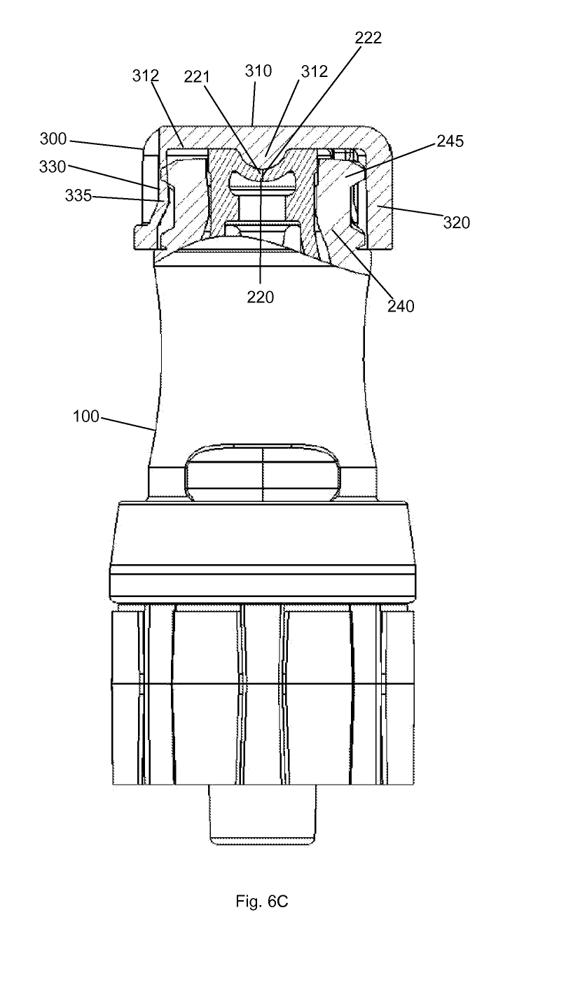

As mentioned above, the cap 300 helps prevent re-knitting of the aperture 220. To that end, as shown in FIGS. 5A-5C, the bottom surface 312 of the body portion 310 may include a distally extending protrusion 315 (e.g., an interacting portion). As best shown in FIGS. 6A and 6B, when the cap 300 is placed on the inlet housing 240 and the clips 330 engage the threads 245, the protrusion 315 contacts the aperture 220, slightly deforms the inlet seal 210 and aperture 220, and separates the aperture's slit planes 221/222 such that they are no longer in contact with one another. By preventing contact between the slit planes 221/222, various embodiments of the present invention substantially are able to prevent the re-knitting of the aperture 220 and ensure proper operation of the valve 10 after sterilization and storage.

Although FIG. 6A shows the cap 300 fully opening the aperture 220, it is important to note that, in accordance with some embodiments, when the cap 300 is attached/secured to the inlet housing 240, the protrusion 315 does need to fully open the aperture 220. For example, as shown in FIG. 6B, the protrusion 315 may deform the inlet seal 210 just enough to separate a portion of the aperture/slit planes 221/222. In such embodiments, either the top 224 of the aperture 220 or the bottom 226 of the aperture 220 may remain closed (e.g., a portion of the slit planes may remain in contact), as shown in FIG. 6B.

Additionally, as shown in FIG. 6C, in some embodiments, the protrusion 315 does not need to open the aperture 220 at all (e.g., the aperture 220 may remain closed when the cap 300 is secured to the valve 10). In such embodiments, the protrusion may deform the inlet seal 210 just enough to produce a radially outward force on the aperture 220 (e.g., on the slit planes 221/222) that reduces the compression on the aperture 220 and slit planes 221/222. The radially outward force may be great enough to prevent the re-knitting of the aperture 220, but not great enough to open the aperture 220.

It is important to note that other embodiments of the present invention may have different structures that interact with the inlet seal 210 and aperture 220 to separate the slit planes and prevent re-knitting. For example, the cap 300 may have a donut shaped member or one or more fingers extending distally from the bottom surface 312 of the body portion 310 at the center and/or away from the center of the body portion 310. Additionally or alternatively, the body portion 310 may be shaped such that the bottom surface 312 is angled distally to form a peak or similar structure near the center of the body portion 310. In such embodiments, the peak or similar structure may interact with/deform the inlet seal 210 to prevent/minimize re-knitting of the slit planes 221/222.

As mentioned above, the medical valve 10 may have an internal valve mechanism 610 that controls fluid flow through the valve 10. In order to protect the fluid flow path through valve 10, this valve mechanism should remain in the closed mode until the luer is connected to the valve inlet. (e.g., it should remain in the closed mode during the sterilization and storage). To that end, various embodiments of the present invention do not activate the valve mechanism (e.g., they do not transition the valve from the closed mode to the open mode).

Any number of valve mechanisms may suffice. For example, as shown in FIG. 7, the valve mechanism 610 may include a moveable cannula 620 and a resilient member 630. The moveable cannula may include a hole 625 (e.g., a transverse hole) that is sealed by the resilient member 630 when the valve is in the closed mode. As the nurse 20 connects the luer, the cannula 620 moves distally within the valve 10 to transition the valve 10 to the open mode. When the cap 300 is secured to the inlet housing 240, the protrusion 315 does not deform the inlet seal 210 enough to cause the cannula 610 to substantially move distally within the valve and transition the valve from the closed mode to the open mode (e.g., the valve 10 remains in the closed mode). In other words, the hole 625 remains sealed by the gland member 630, and the valve 10 remains closed.

Prior to using the valve 10 and connecting the luer to open the valve 10 (e.g., to transfer fluids to/from the patient 30), the nurse 20 (or other operator) removes the cap 300. As mentioned above, the clips 330 engage the threads 245 on the inlet housing 240. Therefore, to remove the cap 300 from the valve, the nurse/operator 20 simply needs to rotate the cap 300 and unscrew the cap 300 from the valve 10. As the nurse/operator 20 begins to rotate/unscrew the cap 300, the engagement portion(s) 335 will follow the threads 245 on the inlet housing 240 and allow the cap 300 to be unscrewed/removed.

Alternatively, the nurse/operator 20 may remove the cap 300 by pulling firmly on the cap 300. As the nurse/operator 20 pulls on the cap 330, the threads 245 on the inlet housing 240 will cause the clips 330/living hinges 350/360/370 to, once again, deform/flex radially outward so that the engaging portions 335 may slide over the threads 245 and the cap 300 may be removed.

Other embodiments of the present invention may be secured to the valve 10 in other ways. For example, the cap 300 may be secured to the valve 10 using an interference fit. An interference fit, sometimes called press fit, is a method of fastening/securing two parts by creating friction between the parts as they are pushed together (e.g., between the skirt 320 or clips 330 and the threads 245). In other words, an interference fit may be created between the clips 330 that remain deformed (or only partially return to their undeformed state) and the threads 245. Additionally or alternatively, the valve 10 may include a latching mechanism (not shown) that secures the cap 300 to the valve 10.

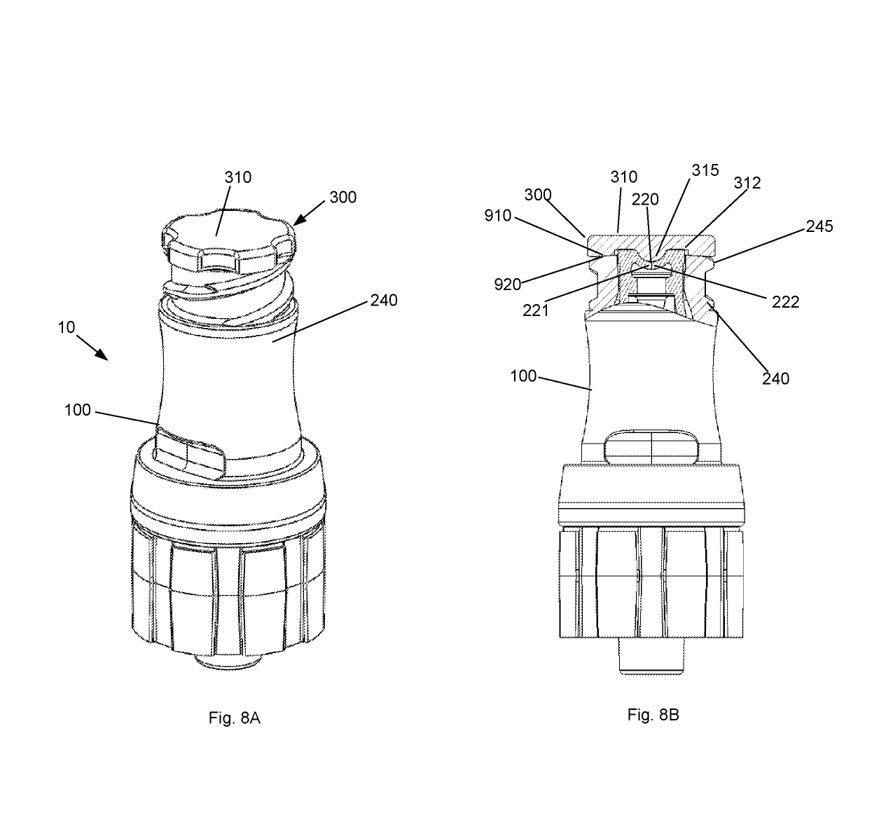

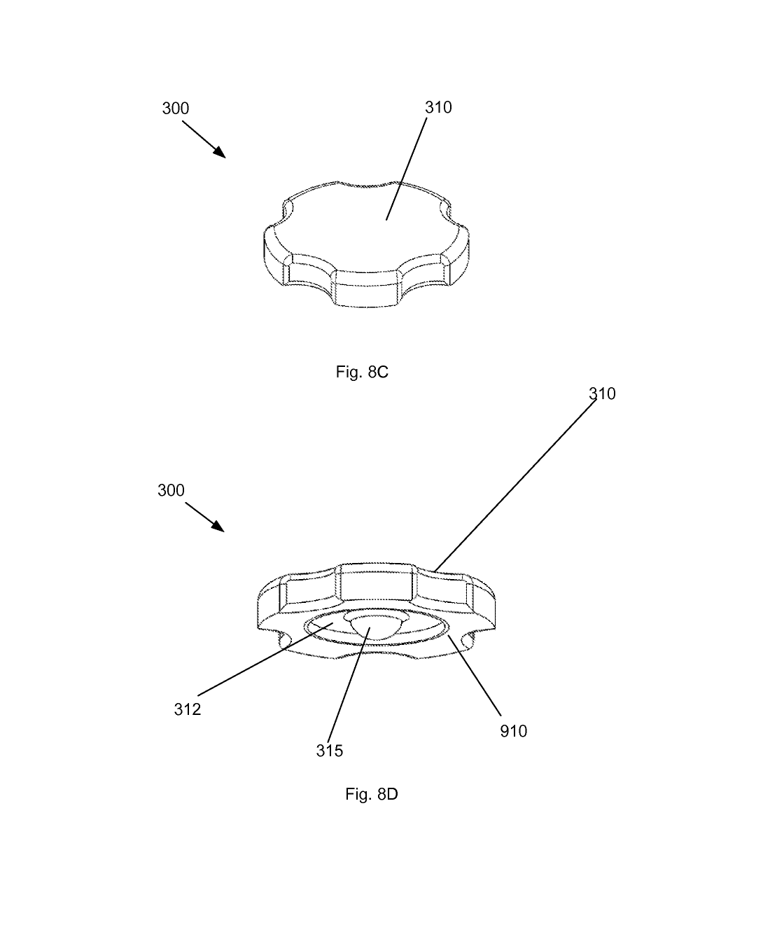

As shown in FIGS. 8A-8D, some embodiments may not use clips, latching mechanisms, or a skirt to secure the cap 300 to the valve 10. For example, the cap 300 may only include the body portion 310 and protrusion/interacting portion 315. In such embodiments, a portion of the underside of the body portion 310 (e.g., a ring 910) may contact the proximal face 920 of the inlet housing 240. The ring 910 may include adhesive (e.g., around the entire ring or just several drops spaced about the diameter of the ring) that secures the cap 300 to the proximal face 920 of the inlet housing 240. In other words, the underside of the body portion 310 (e.g., the ring 910) and the adhesive act as the securing portion that secures the cap 300 to the inlet housing 240.

Like the embodiments described above, when the body portion 310 is secured to the valve 10 using adhesive, the protrusion/interacting portion 315 prevents the aperture 222/inlet seal 210 from re-knitting. It should be noted that, although the adhesive must be strong enough to prevent accidental removal of the cap 300 during sterilization and storage of the valve 10, the adhesive must not be so strong as to prevent the user from removing the cap 300 (e.g., by pulling the cap 300 off of the valve 10) prior to use of the valve 10.

Further embodiments of the cap 300 may have various anti-microbial properties. For example, the cap 300 may contain an impregnated antimicrobial agent or have an antimicrobial coating that maintains a degree of cleanliness at all times. Additionally or alternatively, the bottom surface 312 of the cap 300 may include an alcohol swab or other material containing an antimicrobial agent. In such embodiments, the cap 300 may perform the initial swabbing step as the cap 300 is removed from the valve 10 (e.g., as the cap 300 is twisted to remove it from the valve 10, the swab/material will swab the top surface of the valve 10).

Although the embodiments described above are used with medical valves having a valve mechanism with a moveable cannula 620 and a resilient member 630, other embodiments of the present invention may be used with medical valves having different valve mechanisms. For example, other embodiments may be used to prevent re-knitting on valves having valve mechanisms with stationary post members, rotating members, etc.

Additionally, some embodiments of the present invention may be used to prevent re-knitting of apertures other than slits. For example, some embodiments of the present invention may be used with valves having apertures 220 that are pin-holes and/or apertures 220 that are one or more slits arranged into a cross or a star pattern. In such embodiments, the protrusion 315 will interact with the inlet seal 210 to minimize/prevent contact between the inner wall of the pinhole and/or the multiple slit planes of the cross/star shaped aperture.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A system for preventing re-knitting of an aperture comprising:
a medical valve having an inlet and proximal seal, the proximal seal having a normally closed aperture therethrough; and
a cap removably secured to the inlet and having a body portion and a securing portion, the securing portion securing the cap to the inlet, the body portion having an interacting portion that interacts with the proximal seal to prevent the aperture from re-knitting.

2. A system according to claim 1, wherein the securing portion engages threads located on the valve inlet, thereby securing the cap to the medical valve.

3. A system according to claim 1, wherein the securing portion includes a skirt that extends distally from the body portion and over the inlet of the medical valve.

4. A system according to claim 1, wherein the aperture has a first aperture plane and a second aperture plane, the interacting portion deforming the proximal seal to at least partially separate the first and second aperture planes when the cap is secured to the valve.

5. A system according to claim 1, wherein the interacting portion includes at least one protrusion extending distally from the body portion.

6. A system according to claim 1, wherein the interacting portion opens the aperture when the cap is secured to the valve.

7. A system according to claim 1, further comprising:
a plurality of clips spaced about the securing portion, the plurality of clips having an engaging portion that engages threads located on the valve inlet and secures the cap to the valve.

8. A system according to claim 7, wherein the plurality of clips are configured to deform radially outward, thereby allowing the cap to slide over the inlet of the valve.

9. A system according to claim 8, wherein each of the plurality of clips have at least one living hinge, the at least one living hinge allowing the plurality of clips to deform radially outward.

10. A system according to claim 7, wherein the plurality of clips are configured such that, when secured to the valve, the cap may be unthreaded from the medical valve to remove the cap from the medical valve.

11. A system according to claim 1, wherein the aperture has a first and second aperture plane, the interacting portion urging the aperture planes away from one another as it interacts with the proximal seal.

12. A system according to claim 1, wherein the medical valve includes a valve mechanism within an interior of the valve, the valve mechanism having an open mode which permits fluid flow through the valve and a closed mode that prevents fluid flow through the valve, the valve mechanism remaining in the closed mode as the interacting portion interacts with the proximal seal.

13. A system according to claim 1, wherein the cap has antimicrobial properties.

14. A system according to claim 1, wherein the cap includes an antimicrobial swab that swabs the top of the valve as the cap is removed.

15. A method for preventing re-knitting of an aperture within a medical valve comprising:
providing a medical valve having an inlet housing, an inlet seal with an aperture, and a valve mechanism within an interior of the valve, the valve mechanism configured to transition the valve from a closed mode that prevents fluid flow through the valve to an open mode that permits fluid flow through the valve; and
securing a cap to the inlet housing of the medical valve, the cap including a body portion and a securing portion extending distally from the body portion and over the inlet housing, the body portion having an interacting portion that interacts with the proximal seal to prevent the aperture from re-knitting.

16. A method according to claim 15, wherein the securing portion engages threads located on the inlet housing, thereby securing the cap to the medical valve.

17. A method according to claim 15, wherein the aperture has a first aperture plane and a second aperture plane, the interacting portion deforming the proximal seal to at least partially separate the first and second aperture planes when the cap is connected to the valve.

18. A method according to claim 15, wherein the interacting portion causes the aperture to open when the cap is secured to the valve.

19. A method according to claim 15, wherein the cap further includes a plurality of clips spaced about the securing portion, the plurality of clips having an engaging portion that engages threads located on the valve inlet and securing the cap to the valve.

20. A method according to claim 19, wherein the plurality of clips are configured to deform radially outward, thereby allowing the cap to slide over an inlet of the valve.

21. A cap according to claim 20, wherein each of the plurality of clips have at least one living hinge, the at least one living hinge allowing the plurality of clips to deform radially outward.

22. A method according to claim 19, wherein the plurality of clips are configured such that, when secured to the valve, the cap may be unthreaded from the medical valve to remove the cap from the medical valve.

23. A method according to claim 15, wherein the aperture has a first and second aperture plane, the interacting portion urging the aperture planes away from one another as it interacts with the proximal seal.

24. A method according to claim 15, wherein the medical valve includes a valve mechanism within an interior of the valve, the valve mechanism having an open mode which permits fluid flow through the valve and a closed mode that prevents fluid flow through the valve, the valve mechanism remaining in the closed mode as the interacting portion interacts with the proximal seal.

25. A method according to claim 15, wherein the interacting portion is at least one protrusion extending distally from the body portion.

26. A cap for a medical valve to prevent re-knitting of an aperture within a proximal seal of the medical valve, the cap comprising:
a body portion;
a skirt extending distally from the body portion and configured to fit over an inlet of the medical valve; and
an interacting portion extending distally from the body portion, the interacting portion interacting with the proximal seal to prevent the aperture from re-knitting.

27. A cap according to claim 26, wherein the skirt engages threads located on the valve inlet, thereby securing the cap to the medical valve.

28. A cap according to claim 26, wherein the aperture has a first aperture plane and a second aperture plane, the interacting portion deforming the proximal seal to at least partially separate the first and second aperture planes when the cap is connected to the valve.

29. A cap according to claim 26, wherein the interacting portion causes the aperture to open when the cap is secured to the valve.

30. A cap according to claim 26, further comprising:
a plurality of clips spaced about the skirt, the plurality of clips having an engaging portion that engages threads located on the valve inlet and securing the cap to the valve.

31. A cap according to claim 30, wherein the plurality of clips are configured to deform radially outward, thereby allowing the cap to slide over an inlet of the valve.

32. A cap according to claim 31, wherein each of the plurality of clips have at least one living hinge, the at least one living hinge allowing the plurality of clips to deform radially outward.

33. A cap according to claim 30, wherein the at least one clip is configured such that, when secured to the valve, the cap may be unthreaded from the medical valve to remove the cap from the medical valve.

34. A cap according to claim 26, wherein the aperture has a first and second aperture plane, the interacting portion urging the aperture planes away from one another as it interacts with the proximal seal.

35. A cap according to claim 26, wherein the medical valve includes a valve mechanism within an interior of the valve, the valve mechanism having an open mode which permits fluid flow through the valve and a closed mode that prevents fluid flow through the valve, the valve mechanism remaining in the closed mode as the interacting portion interacts with the proximal seal.

36. A cap according to claim 26, wherein the interacting portion is at least one protrusion extending distally from the body portion.

37. A system for preventing re-knitting of an aperture comprising:
a medical valve having an inlet and proximal seal, the proximal seal having a normally closed aperture with a first slit plane and a second slit plane; and
a cap removably secured to the inlet and having a body portion and a securing portion, the securing portion securing the cap to the inlet, the body portion having an interacting portion that applies a radially outward force upon the proximal seal, thereby preventing re-knitting of the aperture.

38. A system according to claim 37, wherein the securing portion engages threads located on the valve inlet, thereby securing the cap to the medical valve.

39. A system according to claim 37, wherein the securing portion includes a skirt that extends distally from the body portion and over the inlet of the medical valve.

40. A system according to claim 37, wherein the interacting portion deforms the proximal seal to at least partially separate the first and second aperture planes when the cap is secured to the valve.

41. A system according to claim 37, wherein the interacting portion includes at least one protrusion extending distally from the body portion.

42. A system according to claim 37, wherein the radially outward force causes the aperture to open when the cap is secured to the valve.

43. A system according to claim 37, further comprising:
a plurality of clips spaced about the securing portion, the plurality of clips having an engaging portion that engages threads located on the valve inlet and secures the cap to the valve.

44. A system according to claim 43, wherein the plurality of clips are configured to deform radially outward, thereby allowing the cap to slide over the inlet of the valve.

45. A system according to claim 44, wherein each of the plurality of clips has at least one living hinge, the at least one living hinge allowing the plurality of clips to deform radially outward.

46. A system according to claim 43, wherein the plurality of clips are configured such that, when secured to the valve, the cap may be unthreaded from the medical valve to remove the cap from the medical valve.

47. A system according to claim 37, wherein the interacting portion urges the aperture planes away from one another as it interacts with the proximal seal.

48. A system according to claim 37, wherein the medical valve includes a valve mechanism within an interior of the valve, the valve mechanism having an open mode which permits fluid flow through the valve and a closed mode that prevents fluid flow through the valve, the valve mechanism remaining in the closed mode as the interacting portion interacts with the proximal seal.

49. A system according to claim 37, wherein the cap has antimicrobial properties.

50. A system according to claim 37, wherein the cap includes an antimicrobial swab that swabs the top of the valve as the cap is removed.
